(19) United States
(12) Patent Application Publication
(10) Pub. No.: US 2026/0114802 A1
(43) Pub. Date: Apr. 30, 2026

(54) METHOD AND SYSTEM FOR MEASURING RESPIRATORY FLOW BY MEASURING CHANGE IN AIR

(71) Applicant: SB SOLUTIONS INC., Ulsan (KR)

(72) Inventors: Namhwan SUNG, Ulsan (KR); Hyungki MIN, Ulsan (KR); Seungup SEO, Ulsan (KR)

(73) Assignee: SB SOLUTIONS INC., Ulsan (KR)

(21) Appl. No.: 19/432,265

(22) Filed: Dec. 24, 2025

Related U.S. Application Data
(63) Continuation of application No. PCT/KR2023/013203, filed on Sep. 5, 2023.

(30) Foreign Application Priority Data
Sep. 4, 2023 (KR) 10-2023-0116866

(51) Int. Cl. A61B 5/00; A61B 5/08; A61B 5/087; A61B 5/11; A61B 5/145

(52) U.S. Cl. CPC A61B 5/4818; A61B 5/0816; A61B 5/0878; A61B 5/1126; A61B 5/14542; A61B 5/6819; A61B 5/742; A61B 2562/0219; A61B 2562/0271; A61B 2562/029; A61B 2562/166

(57) ABSTRACT

The present disclosure discloses a method and system for measuring respiratory flow by using an air change measurement sensor. A system for measuring respiratory flow according to an embodiment may include an air change measurement sensor disposed around a body organ of a user in which inhalation and exhalation occur according to respiration of the user, and configured to measure a change in air according to the inhalation and the exhalation, and a circuit unit attached to a body of the user and connected to the air change measurement sensor such that the air change measurement sensor is disposed around the body organ, and configured to receive information on the change in air measured by the air change measurement sensor. In this case, respiratory flow of the user may be measured on the basis of the information on the change in air.

### CROSS-REFERENCE TO RELATED APPLICATION(S)

This U.S. non-provisional application is a continuation application of PCT International Application PCT/KR2023/013203, which has an international filing date of Sep. 5, 2023, now published as WO 2025053299A1, which claims the priority benefit of Korean Patent Application No. 10-2023-0116866, filed on Sep. 4, 2023, in the Korean Intellectual Property Office, now granted as Korean Patent No. 10-2721206, the disclosures of which are herein incorporated by reference in its entirety.

### BACKGROUND OF THE INVENTION

### Field of Invention

The following description relates to a method and system for measuring respiratory flow by measuring change in air.

### Description of Related Art

Measurement of respiratory flow is an essential element for diagnosing sleep apnea together with oxygen saturation. Conventional respiratory flow measurement methods use a scheme in which a tube is inserted into a nose or a mask is worn and a pressure of airflow is measured, and thus inconvenience in wearing occurs due to the tube or the mask, and in severe cases, situations occur in which breathing becomes difficult and measurement becomes impossible.

### BRIEF SUMMARY OF THE INVENTION

A method and system for measuring respiratory flow by measuring change in air are provided.

The present disclosure provides a system for measuring respiratory flow, comprising: an air change measurement sensor disposed around a body organ of a user in which inhalation and exhalation occur according to respiration of the user, and configured to measure a change in air according to the inhalation and the exhalation; and a circuit unit attached to a body of the user, connected to the air change measurement sensor such that the air change measurement sensor is disposed around the body organ, and configured to receive information on the change in air measured by the air change measurement sensor, wherein the respiratory flow of the user is measured on the basis of the information on the change in air.

According to one aspect, the change in air according to the inhalation and the exhalation includes at least one of a temperature change and a humidity change of the air according to the inhalation and the exhalation, and the air change measurement sensor may include at least one of a temperature sensor and a humidity sensor.

According to other aspect, the circuit unit may measure the respiratory flow of the user on the basis of the information on the change in air.

According to still other aspect, the circuit unit may transmit the information on the change in air to an external device for measuring the respiratory flow of the user.

According to still other aspect, at least one of a respiration rate of the user and whether the user has apnea may be determined on the basis of the respiratory flow in the circuit unit or in an external device communicating with the circuit unit.

According to still other aspect, the air change measurement sensor may include a flexible printed circuit board (Flexible PCB), and the air change measurement sensor may be adjusted through the flexible PCB to be disposed around the body organ of the user.

According to still other aspect, the circuit unit may be attached to an outer surface of a nose of the user, and the air change measurement sensor may be connected to the circuit unit along a curvature of the nose of the user and disposed in front of the nose of the user.

According to still other aspect, the circuit unit may transmit, to an external device, at least one of the information on the change in air, the respiratory flow, a respiration rate of the user determined on the basis of the respiratory flow, and whether the user has apnea determined on the basis of the respiratory flow, and the external device may display, through a display, at least one of the information on the change in air, the respiratory flow, the respiration rate of the user determined on the basis of the respiratory flow, and whether the user has apnea determined on the basis of the respiratory flow.

According to still other aspect, the respiratory flow measurement system may further include at least one sensor selected from an oxygen saturation sensor for measuring oxygen saturation of the user and a motion sensor for measuring motion of the user.

The present disclosure further provides a respiratory flow measurement method of a respiratory flow measurement system, the method comprising: operating, in a circuit unit included in the respiratory flow measurement system, an air change measurement sensor further included in the respiratory flow measurement system to measure a change in air according to inhalation and exhalation occurring according to respiration of a user; transmitting, in the circuit unit, information on the change in air measured by the air change measurement sensor to an external device, or measuring, in the circuit unit, respiratory flow of the user on the basis of the information on the change in air; and when the respiratory flow is measured in the circuit unit, transmitting, in the circuit unit, information on the measured respiratory flow to the external device.

### BRIEF DESCRIPTION OF THE DRAWINGS

FIG. 1 is a diagram illustrating an example of an overall configuration of a system for measuring respiratory flow according to an embodiment of the present disclosure.

FIG. 2 is a diagram illustrating an example of temperature change data and respiratory flow data according to an embodiment of the present disclosure.

FIG. 3 is a diagram illustrating an example of an internal configuration of the system for measuring respiratory flow according to an embodiment of the present disclosure.

FIG. 4 is a flowchart illustrating an example of a method for measuring respiratory flow according to an embodiment of the present disclosure.

FIG. 5 is a block diagram illustrating an example of a computer device according to an embodiment of the present disclosure.

### DETAILED DESCRIPTION OF THE INVENTION

Hereinafter, embodiments will be described in detail with reference to the accompanying drawings. However, various modifications may be made to the embodiments, and the scope of the claims of the patent application is not limited or restricted by these embodiments. All modifications, equivalents, and alternatives to the embodiments should be understood to be included within the scope of the claims.

The terms used in the embodiments are used for descriptive purposes only and should not be construed as limiting. Singular expressions include plural expressions unless the context clearly indicates otherwise. In the present specification, terms such as “comprise” or “have” are intended to designate that the features, numbers, steps, operations, components, parts, or combinations thereof described in the specification exist, and should be understood as not precluding in advance the existence or possibility of addition of one or more other features, numbers, steps, operations, components, parts, or combinations thereof.

Unless otherwise defined, all terms used herein, including technical and scientific terms, have the same meaning as commonly understood by a person having ordinary skill in the art to which the embodiments pertain. Such terms as are defined in a dictionary of common use shall be interpreted as having a meaning that is consistent with the meaning they have in the context of the relevant technology and shall not be interpreted as having an ideal or excessively formal meaning unless expressly defined in the present application.

In addition, in describing the embodiments with reference to the accompanying drawings, the same components are denoted by the same reference numerals regardless of the drawing numerals, and redundant descriptions thereof will be omitted. In describing the embodiments, when it is determined that a detailed description of related known technologies may unnecessarily obscure the gist of the embodiments, such detailed description will be omitted.

In addition, in describing components of the embodiments, terms such as first, second, A, B, (a), and (b) may be used. These terms are used only for distinguishing one component from another component, and do not limit the nature, order, or sequence of the corresponding components by such terms. When a component is described as being “connected,” “coupled,” or “linked” to another component, it should be understood that the component may be directly connected or linked to the other component, or another component may be connected, coupled, or linked between the respective components.

Components included in one embodiment and components including common functions will be described using the same names in other embodiments. Unless otherwise stated, descriptions described in one embodiment may be applied to other embodiments, and detailed descriptions thereof will be omitted to the extent of overlap.

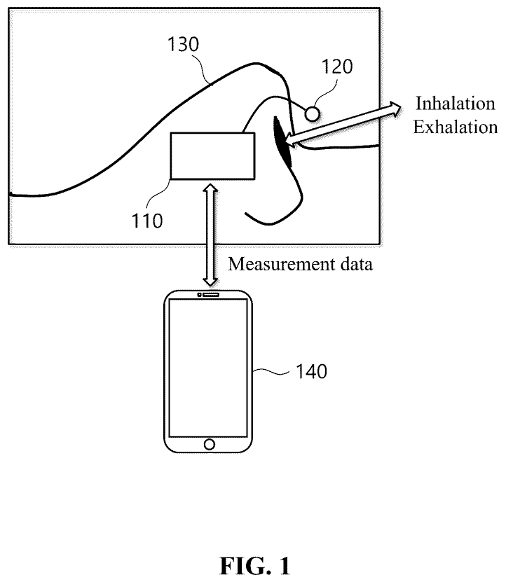

FIG. 1 is a diagram illustrating an example of an overall configuration of a system for measuring respiratory flow according to an embodiment of the present disclosure. The system for measuring respiratory flow according to the present embodiment may include a circuit unit 110 and an air change measurement sensor 120.

The circuit unit 110 may be attached to a body of a user, and the air change measurement sensor 120 may be disposed around a body organ of the user in which inhalation and exhalation occur according to respiration of the user. In the embodiment of FIG. 1, an example is illustrated in which the circuit unit 110 is attached to an outer surface of a nose 130 of the user, and the air change measurement sensor 120 connected to the circuit unit 110 along a curvature of the nose of the user is disposed in front of the nose 130 of the user (for example, in front of an external nostril of the nose 130). According to embodiments, the circuit unit 110 may be attached to a body part of the user other than the outer surface of the nose 130, such as a cheek of the user. In addition, the air change measurement sensor 120 may be disposed in front of a mouth of the user rather than in front of the nose 130 of the user.

In this case, the air change measurement sensor 120 may measure a change in air according to inhalation and exhalation occurring according to respiration of the user, such as a temperature change of air and/or a humidity change of air. To this end, the air change measurement sensor 120 may include a temperature sensor and/or a humidity sensor. For example, the air change measurement sensor 120 may continuously measure a change in air over time. In this case, the circuit unit 110 may receive and accumulate information on the change in air measured by the air change measurement sensor 120 from the air change measurement sensor 120. Hereinafter, the accumulated information on the change in air (temperature change and/or humidity change) will be referred to as air change data. The air change data may include temperature change data and/or humidity change data.

Respiratory flow of the user may be obtained through the air change data. For example, respiratory flow of the user may be measured as a pattern of inhalation and exhalation of the user through a pattern of a temperature change obtained from the temperature change data, or respiratory flow of the user may be measured as a pattern of inhalation and exhalation of the user through a pattern of a humidity change obtained from the humidity change data. According to embodiments, respiratory flow of the user may be measured as a pattern of inhalation and exhalation of the user by using both the pattern of the temperature change and the pattern of the humidity change. Hereinafter, information on accumulated respiratory flow will be referred to as respiratory flow data. As described above, by using the system for measuring respiratory flow according to the present embodiment, respiratory flow of the user may be measured by measuring a temperature change and/or a humidity change by positioning a precise temperature sensor and/or a precise humidity sensor capable of detecting minute temperature and humidity changes in front of the nose 130 of the user. Accordingly, since a tube is not inserted into the user and the user does not wear a mask, inconvenience to the user may be alleviated compared to conventional respiratory flow measurement methods, while respiratory flow of the user may be accurately measured.

The circuit unit 110 may communicate with an external device 140 to transmit measurement data to the external device 140. In this case, the external device 140 may display, on a display, at least one of a waveform graph of air change data (a waveform graph of temperature change data and/or a waveform graph of humidity change data), a waveform graph of respiratory flow data, a respiration rate of the user, and whether the user has apnea, on the basis of the received measurement data, and may provide an alarm to the user according to the respiration rate of the user and/or whether the user has apnea.

In a first embodiment, the circuit unit 110 may generate respiratory flow data by using the air change data. In this case, the circuit unit 110 may transmit the air change data and the respiratory flow data to the external device 140. In this case, the external device 140 may determine the respiration rate of the user and/or whether the user has apnea by using the received respiratory flow data.

In a second embodiment, the circuit unit 110 may transmit the air change data directly to the external device 140. In this case, the external device 140 may generate respiratory flow data by using the air change data, and may determine the respiration rate of the user and/or whether the user has apnea by using the respiratory flow data.

In a third embodiment, the circuit unit 110 may generate respiratory flow data by using the air change data, and may determine the respiration rate of the user and/or whether the user has apnea by using the respiratory flow data. In this case, the circuit unit 110 may transmit, to the external device 140, information on the air change data, the respiratory flow data, and the respiration rate of the user and/or whether the user has apnea.

In each embodiment, the external device 140 may display, on a display included in the external device 140, at least one of a waveform graph of air change data, a waveform graph of respiratory flow data, a respiration rate of the user, and whether the user has apnea, and may provide an alarm to the user according to the respiration rate of the user and/or whether the user has apnea.

In the embodiment of FIG. 1, the external device 140 is illustrated in the form of a smartphone; however, the external device 140 may be implemented in various forms of electronic devices, such as a smartwatch or a tablet PC. In addition, the external device 140 may be connected to the circuit unit 110 by using various wired and/or wireless communication means.

In addition, the air change measurement sensor 120 may include at least one temperature sensor selected from various temperature sensors, such as a capacitive temperature sensor, a digital temperature sensor, and a thermistor. In addition, the air change measurement sensor 120 may include at least one humidity sensor selected from various humidity sensors, such as a capacitive humidity sensor, a resistive humidity sensor, and a thermal-type humidity sensor.

Further, the air change measurement sensor 120 may include a flexible printed circuit board (Flexible PCB), and the air change measurement sensor 120 may be adjusted through the flexible PCB to be disposed around a body organ of the user. For example, when the circuit unit 110 is attached to an outer surface of a nose 130 of the user as in the embodiment of FIG. 1, the air change measurement sensor 120 may be connected to the circuit unit 110 along a curvature of the nose 130 of the user and disposed in front of the nose 130 of the user.

Meanwhile, the circuit unit 110 and/or a connection portion connecting the circuit unit 110 and the air change measurement sensor 120 (for example, a connection line based on a flexible PCB) may be attached to the body of the user by using an attachment means such as tape in order to prevent a positional change of the air change measurement sensor 120.

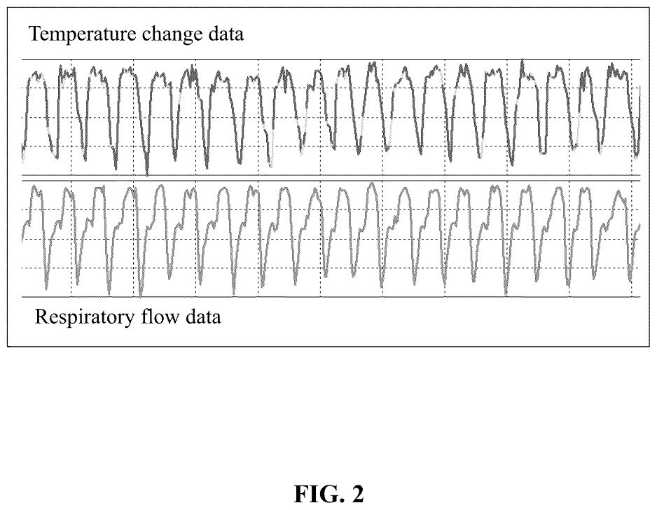

FIG. 2 is a diagram illustrating an example of temperature change data and respiratory flow data according to an embodiment of the present disclosure. In FIG. 2, temperature change data obtained by measuring and accumulating a temperature change of air according to inhalation and exhalation occurring in a body organ of a user by using a temperature sensor, and respiratory flow data measured by a conventional respiratory flow measurement method are respectively illustrated. FIG. 2 shows that a pattern of the temperature change data and a pattern of the respiratory flow data substantially coincide with each other, and accordingly indicates that respiratory flow of the user may be measured by using the temperature change data. Humidity change data obtained by measuring and accumulating a humidity change of air by using a humidity sensor also exhibits a pattern similar to that of the respiratory flow data.

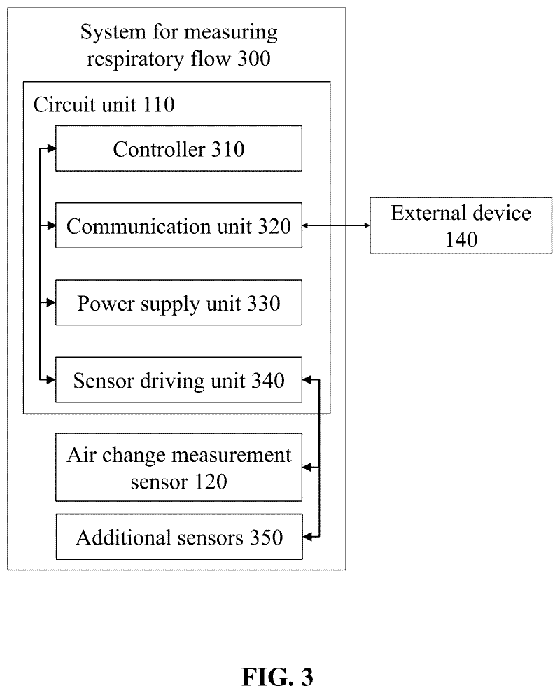

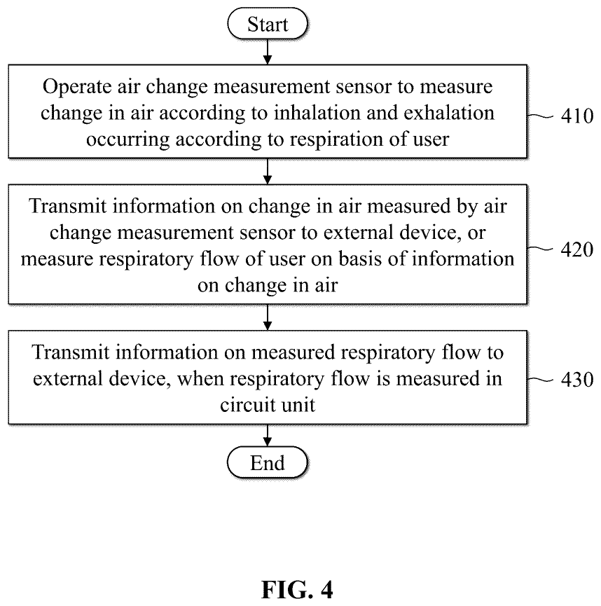

FIG. 3 is a diagram illustrating an example of an internal configuration of a system for measuring respiratory flow according to an embodiment of the present disclosure, and FIG. 4 is a flowchart illustrating an example of a method for measuring respiratory flow according to an embodiment of the present disclosure. As described above, a system for measuring respiratory flow 300 according to the present embodiment may include a circuit unit 110 and an air change measurement sensor 120. In this case, as illustrated in FIG. 3, the circuit unit 110 may include a controller 310, a communication unit 320, a power supply unit 330, and a sensor driving unit 340. The power supply unit 330 may provide power required for the controller 310, the communication unit 320, and the sensor driving unit 340, and the controller 310 may control operations of the communication unit 320, the power supply unit 330, and the sensor driving unit 340. In addition, an application associated with the system for measuring respiratory flow 300 may be installed and executed in the external device 140. In this case, under control of the application, the external device 140 may communicate with the communication unit 320 of the circuit unit 110 to receive measurement data, and may display, on a display, at least one of a waveform graph of air change data (temperature change data and/or humidity change data), a waveform graph of respiratory flow data, a respiration rate of the user, and whether the user has apnea on the basis of the received measurement data, and may provide an alarm to the user according to the respiration rate of the user and/or whether the user has apnea.

In step 410, the circuit unit 110 may operate the air change measurement sensor 120 to measure a change in air according to inhalation and exhalation occurring according to respiration of a user, such as a temperature change of air and/or a humidity change of air. To this end, the air change measurement sensor 120 may include a temperature sensor and/or a humidity sensor. For example, as described above, the circuit unit 110 may be attached to a body of the user, and the air change measurement sensor 120 may be disposed around a body organ of the user (for example, a nose or a mouth of the user) in which inhalation and exhalation occur, in a state of being connected to the circuit unit 110. In a more detailed example, a controller 310 included in the circuit unit 110 may control the air change measurement sensor 120 through a sensor driving unit 340 to continuously measure a change in air, and in this case, a change in air according to inhalation and exhalation may be measured through the air change measurement sensor 120. Information on the change in air measured by the air change measurement sensor 120 may be transmitted to the controller 310 through the sensor driving unit 340.

In step 420, the circuit unit 110 may transmit information on a change in air measured by the air change measurement sensor 120 to an external device 140, or may measure respiratory flow of a user on the basis of the information on the change in air. For example, a first algorithm for measuring respiratory flow may be included in the circuit unit 110 or may be included in the external device 140. When the first algorithm is included in the external device 140, the circuit unit 110 may transmit the information on the change in air measured by the air change measurement sensor 120 to the external device 140 through the communication unit 320. In this case, the external device 140 may measure respiratory flow of the user by using the transmitted information on the temperature change and the first algorithm. When the first algorithm is included in the circuit unit 110, the circuit unit 110 may measure respiratory flow of the user by using the first algorithm and the information on the change in air measured by the air change measurement sensor 120. The first algorithm may include an algorithm for generating respiratory flow data on the basis of air change data in which changes in air measured by the air change measurement sensor 120 are accumulated. As described above, the air change data may include temperature change data and/or humidity change data.

In step 430, when respiratory flow is measured in the circuit unit 110, the circuit unit 110 may transmit information on the measured respiratory flow to the external device 140. In this case, the external device 140 may include a second algorithm for determining a respiration rate of the user and/or whether the user has apnea on the basis of respiratory flow data of the user. In this case, the external device 140 may determine the respiration rate of the user and/or whether the user has apnea by using the respiratory flow of the user and the second algorithm.

Further, according to embodiments, when the first algorithm is included in the circuit unit 110, the second algorithm may also be included in the circuit unit 110. In this case, the circuit unit 110 may determine a respiration rate of the user and/or whether the user has apnea by using respiratory flow data obtained through the first algorithm and the second algorithm. In this case, the circuit unit 110 may transmit, to the external device 140, information on air change data, respiratory flow data, and the respiration rate of the user and/or whether the user has apnea.

As described above, in each embodiment, the external device 140 may display, on a display included in the external device 140, at least one of a waveform graph of air change data, a waveform graph of respiratory flow data, a respiration rate of the user, and whether the user has apnea, and may provide an alarm to the user according to the respiration rate of the user and/or whether the user has apnea. For example, the external device 140 may output the alarm in various manners, such as light, sound, and/or vibration.

Further, according to embodiments, the system for measuring respiratory flow 300 may further include additional sensors 350, such as an oxygen saturation sensor for measuring oxygen saturation of the user and/or a motion sensor for measuring motion of the user. Oxygen saturation and/or motion of the user measured through the additional sensors 350 may be further utilized to determine respiratory flow of the user, a respiration rate of the user, and/or whether the user has apnea. For example, the motion sensor may include a gyroscope and/or an accelerometer.

Communication between the communication unit 320 and the external device 140 may be performed by using at least one of various well-known communication protocols, such as Bluetooth Low Energy (BLE), Near Field Communication (NFC), and WiFi.

Meanwhile, the external device 140 may be implemented, for example, by a computer device.

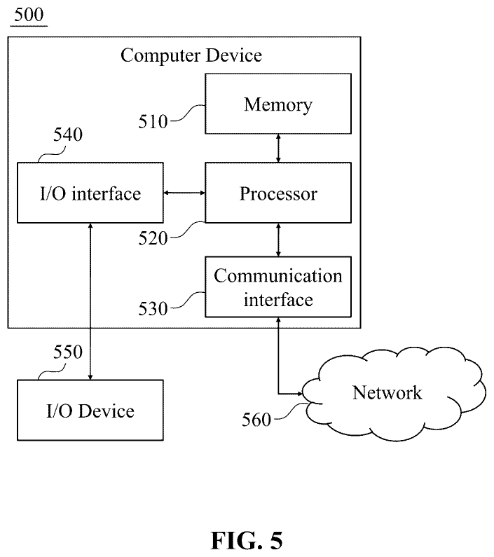

FIG. 5 is a block diagram illustrating an example of a computer device according to an embodiment of the present disclosure. As illustrated in FIG. 5, a computer device 500 may include a memory 510, a processor 520, a communication interface 530, and an input/output (I/O) interface 540. The memory 510 may be a computer-readable recording medium and may include random access memory (RAM), read only memory (ROM), and a permanent mass storage device such as a disk drive. Here, permanent mass storage devices such as the ROM and the disk drive may be included in the computer device 500 as separate permanent storage devices distinguished from the memory 510. In addition, the memory 510 may store an operating system and at least one program code. Such software components may be loaded into the memory 510 from a computer-readable recording medium separate from the memory 510. Such a separate computer-readable recording medium may include a computer-readable recording medium such as a floppy drive, a disk, a tape, a DVD/CD-ROM drive, and a memory card. In another embodiment, the software components may be loaded into the memory 510 through the communication interface 530 rather than through a computer-readable recording medium. For example, the software components may be loaded into the memory 510 of the computer device 500 on the basis of a computer program installed by files received through a network 560.

The processor 520 may be configured to process instructions of a computer program by performing basic arithmetic, logic, and input/output operations. The instructions may be provided to the processor 520 by the memory 510 or the communication interface 530. For example, the processor 520 may be configured to execute instructions received according to program code stored in a recording device such as the memory 510.

The communication interface 530 may provide a function for enabling the computer device 500 to communicate with other devices through the network 560. For example, requests, instructions, data, files, and the like generated by the processor 520 of the computer device 500 according to program code stored in a recording device such as the memory 510 may be transmitted to other devices through the network 560 under control of the communication interface 530. Conversely, signals, instructions, data, files, and the like from other devices may be received by the computer device 500 through the communication interface 530 of the computer device 500 via the network 560. Signals, instructions, data, and the like received through the communication interface 530 may be delivered to the processor 520 or the memory 510, and files and the like may be stored in a storage medium (the above-described permanent storage device) that may be further included in the computer device 500.

The input/output (I/O) interface 540 may be a means for interfacing with an I/O device 550. For example, an input device may include devices such as a microphone, a keyboard, or a mouse, and an output device may include devices such as a display or a speaker. As another example, the input/output interface 540 may be a means for interfacing with a device in which functions for input and output are integrated into a single device, such as a touchscreen. The input/output device 550 may be configured as a single device together with the computer device 500.

Further, in other embodiments, the computer device 500 may include fewer or more components than the components illustrated in FIG. 5. However, there is no need to clearly illustrate most conventional components. For example, the computer device 500 may be implemented to include at least some of the above-described input/output devices 550, or may further include other components such as a transceiver or a database.

As described above, according to embodiments of the present disclosure, a method and system for measuring respiratory flow by measuring a change in air may be provided. The method and system for measuring respiratory flow according to embodiments of the present disclosure may measure respiratory flow of a user on the basis of a change in air according to inhalation and exhalation occurring according to respiration of the user by using an air change measurement sensor and/or a humidity sensor, such as a temperature change of air and/or a humidity change of air, and thus may alleviate inconvenience to the user since a tube is not inserted into the user or the user does not wear a mask.

The system or apparatus described above may be implemented by hardware components, or by a combination of hardware components and software components. For example, the apparatuses and components described in the embodiments may be implemented by using one or more general-purpose computers or special-purpose computers, such as a processor, a controller, an arithmetic logic unit (ALU), a digital signal processor (DSP), a microcomputer, a field programmable gate array (FPGA), a programmable logic unit (PLU), a microprocessor, or any other device capable of executing and responding to instructions. The processing device may execute an operating system (OS) and one or more software applications executed on the operating system. In addition, the processing device may access, store, manipulate, process, and generate data in response to execution of software. For convenience of understanding, a case in which one processing device is used has been described; however, those skilled in the art will appreciate that the processing device may include a plurality of processing elements and/or a plurality of types of processing elements. For example, the processing device may include a plurality of processors, or may include one processor and one controller. In addition, other processing configurations, such as a parallel processor, are also possible.

Software may include a computer program, code, instructions, or a combination of one or more thereof, and may configure a processing device to operate as desired or may instruct the processing device independently or collectively. Software and/or data may be embodied in any type of machine, component, physical device, virtual equipment, computer storage medium, or device in order to be interpreted by a processing device or to provide instructions or data to the processing device. Software may be distributed over network-connected computer systems and may be stored or executed in a distributed manner. Software and data may be stored in one or more computer-readable recording media.

Although the embodiments have been described above with reference to limited embodiments and drawings, those skilled in the art will appreciate that various modifications and variations are possible based on the above description. For example, the described techniques may be performed in an order different from the described order, and/or components of the described systems, structures, apparatuses, circuits, and the like may be combined or assembled in a form different from the described form, or may be replaced or substituted by other components or equivalents, while appropriate results may still be achieved.

Accordingly, other implementations, other embodiments, and equivalents to the claims also fall within the scope of the claims described below.

What is claimed is:

1. A system for measuring respiratory flow, comprising:
an air change measurement sensor disposed around a body organ of a user in which inhalation and exhalation occur according to respiration of the user, and configured to measure a change in air according to the inhalation and the exhalation; and
a circuit unit attached to a body of the user, connected to the air change measurement sensor such that the air change measurement sensor is disposed around the body organ, and configured to receive information on the change in air measured by the air change measurement sensor,
wherein the respiratory flow of the user is measured on the basis of the information on the change in air,
wherein the change in air according to the inhalation and the exhalation includes a temperature change and a humidity change of the air according to the inhalation and the exhalation,
wherein the air change measurement sensor comprises a temperature sensor configured to measure the temperature change and a humidity sensor configured to measure the humidity change,
wherein the circuit unit is attached to an outer surface of a nose of the user, and
wherein the air change measurement sensor is connected along a curvature of the nose of the user to the circuit unit and is disposed, in a state including the temperature sensor and the humidity sensor, in front of an external nostril of the nose of the user and spaced outward from the external nostril, such that the change in air is measurable without inserting a tube into the nose of the user or the user wearing a mask.

2. The system for measuring respiratory flow according to claim 1, wherein
the circuit unit measures the respiratory flow of the user on the basis of the information on the change in air.

3. The system for measuring respiratory flow according to claim 1, wherein
the circuit unit transmits the information on the change in air to an external device for measuring the respiratory flow of the user.

4. The system for measuring respiratory flow according to claim 1, wherein
at least one of a respiration rate of the user and whether the user has apnea is determined on the basis of the respiratory flow in the circuit unit or in an external device communicating with the circuit unit.

5. The system for measuring respiratory flow according to claim 1, wherein
the air change measurement sensor comprises a flexible printed circuit board (Flexible PCB), and
the air change measurement sensor is adjusted through the flexible PCB to be disposed around the body organ of the user.

6. The system for measuring respiratory flow according to claim 1, wherein
the circuit unit transmits, to an external device, at least one of the information on the change in air, the respiratory flow, a respiration rate of the user determined on the basis of the respiratory flow, and whether the user has apnea determined on the basis of the respiratory flow, and
the external device displays, through a display, at least one of the information on the change in air, the respiratory flow, the respiration rate of the user determined on the basis of the respiratory flow, and whether the user has apnea determined on the basis of the respiratory flow.

7. The system for measuring respiratory flow according to claim 1, further comprising:
at least one sensor selected from an oxygen saturation sensor for measuring oxygen saturation of the user and a motion sensor for measuring motion of the user.

8. A method for measuring respiratory flow, performed by a system for measuring respiratory flow, the method comprising:
measuring, by an air change measurement sensor disposed around a body organ of a user in which inhalation and exhalation occur according to respiration of the user, a change in air according to the inhalation and the exhalation;
receiving, by a circuit unit attached to a body of the user and connected to the air change measurement sensor, information on the change in air measured by the air change measurement sensor; and
measuring respiratory flow of the user on the basis of the information on the change in air,
wherein the change in air according to the inhalation and the exhalation includes a temperature change and a humidity change of the air according to the inhalation and the exhalation,
wherein the air change measurement sensor comprises a temperature sensor configured to measure the temperature change and a humidity sensor configured to measure the humidity change,
wherein the circuit unit is attached to an outer surface of a nose of the user, and
wherein the air change measurement sensor is connected along a curvature of the nose of the user to the circuit unit and is disposed, in a state including the temperature sensor and the humidity sensor, in front of an external nostril of the nose of the user and spaced outward from the external nostril, such that the change in air is measurable without inserting a tube into the nose of the user or the user wearing a mask.
